(19) United States
(12) Patent Application Publication
(10) Pub. No.: US 2013/0267763 A1
(43) Pub. Date: Oct. 10, 2013

(54) TRANSVERSE TRANSCRANIAL MAGNETIC STIMULATION COIL PLACEMENT FOR IMPROVED ANALGESIA

(71) Applicants: M. Bret Schneider, Portola Valley, CA (US); David J. Mishelevich, Playa del Rey, CA (US)

(72) Inventors: M. Bret Schneider, Portola Valley, CA (US); David J. Mishelevich, Playa del Rey, CA (US)

(73) Assignee: CERVEL NEUROTECH, INC., San Mateo, CA (US)

(21) Appl. No.: 13/877,428

(22) PCT Filed: Oct. 10, 2011
(86) PCT No.: PCT/US2011/055594

§ 371 Date: Jun. 27, 2013

Related U.S. Application Data
(60) Provisional application No. 61/391,552, filed on Oct. 8, 2010.

(51) Int. Cl. A61N 2/00

(52) U.S. Cl. CPC A61N 2/006
USPC 600/13

(57) ABSTRACT

Described herein are methods for neuromodulating brain activity of one or more target brain regions, the methods using Transcranial Magnetic Stimulation (TMS) to produce robust analgesia. In particular, described herein are systems for arranging one or more (e.g., a plurality) of TMS electromagnets oriented in a transverse direction, perpendicular to the posterior-anterior axis of the head, and applying sufficient energy to neuromodulate the target deep brain region.

### CROSS REFERENCE TO RELATED APPLICATIONS

This patent application claims priority to provisional patent application No. 61/391,552, titled “TRANSVERSE TRANSCRANIAL MAGNETIC STIMULATION COIL PLACEMENT FOR IMPROVED ANALGESIA” and filed on Oct. 8, 2010.

This patent application may be related to one or more of the following patents and pending patent applications (US and PCT applications), each of which is herein incorporated by reference in its entirety: U.S. Pat. No. 7,520,848, titled “ROBOTIC APPARATUS FOR TARGETING AND PRODUCING DEEP, FOCUSED TRANSCRANIAL MAGNETIC STIMULATON”, issued on Apr. 21, 2009; U.S. patent application Ser. No. 12/402,404, titled “ROBOTIC APPARATUS FOR TARGETING AND PRODUCING DEEP, FOCUSED TRANSCRANIAL MAGNETIC STIMULATON”, filed on Mar. 11, 2009; U.S. patent application Ser. No. 11/429,504, titled “TRAJECTORY-BASED DEEP-BRAIN STEREOTACTIC TRANSCRANIAL MAGNETIC STIMULATION”, filed on May 5, 2006; U.S. patent application Ser. No. 12/669,882, titled “DEVICE AND METHOD FOR TREATING HYPERTENSION VIA NON-INVASIVE NEUROMODULATION”, filed on Jan. 20, 2010; U.S. patent application Ser. No. 12/671,260, titled “GANTRY AND SWITCHES FOR POSITION-BASED TRIGGERING OF TMS PULSES IN MOVING COILS”, filed on Jan. 29, 2010; U.S. patent application Ser. No. 12/670,938, titled “FIRING PATTERNS FOR DEEP BRAIN TRANSCRANIAL MAGNETIC STIMULATION”, filed on Jan. 27, 2010; U.S. patent application Ser. No. 12/677,220, titled “FOCUSED MAGNETIC FIELDS”, filed on Mar. 9, 2010; PCT Application No. PCT/US2008/077851, titled “SYSTEMS AND METHODS FOR COOLING ELECTROMAGNETS FOR TRANSCRANIAL MAGNETIC STIMULATION”, filed on Sep. 26, 2008; PCT Application No. PCT/US2008/081048, titled “INTRA-SESSION CONTROL OF TRANSCRANIAL MAGNETIC STIMULATION”, filed on Oct. 24, 2008; U.S. patent application Ser. No. 12/324,227, titled “TRANSCRANIAL MAGNETIC STIMULATION OF DEEP BRAIN TARGETS”, filed on Nov. 26, 2008; PCT Application No. PCT/US2009/045109, titled “TRANSCRANIAL MAGNETIC STIMULATION BY ENHANCED MAGNETIC FIELD PERTURBATIONS”, filed on May 26, 2009; U.S. patent application Ser. No. 12/185,544, titled “MONOPHASIC MULTI-COIL ARRAYS FOR TRANSCRANIAL MAGNETIC STIMULATION”, filed on Aug. 4, 2008; U.S. patent application Ser. No. 12/701,395, titled “CONTROL AND COORDINATION OF TRANSCRANIAL MAGNETIC STIMULATION ELECTROMAGNETS FOR MODULATION OF DEEP BRAIN COILS FOR TRANSCRANIAL MAGNETIC STIMULATION”, filed on Jan. 7, 2010; and U.S. patent application Ser. No. 12/838,299 TRANSCRANIAL MAGNETIC STIMULATION FIELD SHAPING, filed on Jul. 16, 2010.

### INCORPORATION BY REFERENCE

All publications, including patents and patent applications, mentioned in this specification are herein incorporated by reference in their entirety to the same extent as if each individual publication was specifically and individually indicated to be incorporated by reference.

### FIELD

Described herein are systems and methods for modulating brain targets so as to produce analgesia using low-frequency Transcranial Magnetic Stimulation (TMS).

### BACKGROUND

Repetitive transcranial magnetic stimulation (rTMS) involves placing an electromagnetic coil on the scalp while high-intensity current is rapidly turned on and off in the coil through the discharge of capacitors. This produces a time-varying magnetic field that lasts for about 100 to 200 microseconds. The magnetic field is typically about 2 Tesla. The proximity of the brain to the time-varying magnetic field results in current flow in neural tissue. Thus, rTMS provides a powerful opportunity for non-invasive stimulation of superficial cerebral cortex in both healthy subjects and those with a range of psychiatric or neurological disorders. Primarily, however, rTMS stimulation studies have focused on the stimulation superficial cortex, and observing secondary effects in deeper regions of the brain. This is because conventional TMS device designs have been unable to focally modulate subcortical regions directly without overwhelming superficial cortex.

In the early 1990s, Mark George and colleagues described the antidepressant effect of rTMS when applied to the left dorsolateral prefrontal cortex. Since that time, rTMS has become a recognized as an effective method for treating depression. One rTMS device (NeuroStar system by Neuronetics Inc, Malvern, Pa.) has received FDA clearance for marketing for the treatment of depression.

Jean-Pascal Lefaucheur and colleagues have examined repetitive Transcranial Magnetic Stimulation (rTMS) of the motor (pre-central) cortex for pain relief (Lefaucheur, J.-P., Drouot, X., Keravel, Y., and J.-P. Nguyen, “Pain relief induced by repetitive transcranial magnetic stimulation of precentral cortex,” Neuroreport: 17 Sep. 2001, 12:13, pp. 2963-2965, and Lefaucheur, J.-P., Hatem, S., Nineb, A., Ménard-Lefaucheur, I., Wendling, S., Keravel, Y., and J.-P. Nguyen, “Somatotopic organization of the analgesic effects of motor cortex rTMS in neuropathic pain,” Neurology 67:1998-2004, 2006). Lefaucheur (“Use of repetitive transcranial magnetic stimulation in pain relief,” Expert Review of Neurotherapeutics, May 2008, Vol. 8, No. 5, Pages 799-808, DOI 10.1586/14737175.8.5.799 (doi:10.1586/14737175.8.5.799) notes that a subset of patients will get relief from rTMS but relapse and for those patients surgically implanted epidural cortical electrodes and associated pulse generator can be proposed to allow pain relief more permanent, and that the rate of improvement due to rTMS may be predictive of the outcome of such an implantation.

In the medical literature, TMS coils are, by convention, almost always positioned with their handles pointing straight back, away from the face of the patient. This may also be referred to positioning along an anterior/posterior axis. In this position, the majority of the electric conventional current induced within the underlying brain will move from the back of the patient's head, toward the front of the patient's head, in line with the anterior/posterior (A/P) axis of the head. Standard coil positioning for the treatment of depression using TMS is accomplished in this manner in which induced current along the anterior-posterior axis predominates. Further, most conventional TMS reaches only the superficial cortical regions, and it is further uncertain how to orient one or more TMS coils when applying stimulation to deep brain targets.

In the article “Pain relief by rTMS: Differential effect of current flow but no specific action on pain subtypes” (Andre-Obadia N, Mertens P, Gueguen A, Peyron R, Garcia-Larrea L. Neurology 2008; 71:833-840), Andre-Obadia and colleagues test a “lateral-medial” (LM) coil position for a single coil over motor cortex region of the brain of pain patients. This “latero-medial position” is equivalent to the “transverse” positioning discussed herein. The authors conclude that this position is inferior to standard posterior-anterior (PA) positioning of the coil for producing analgesia, the latter of which produced a mean of 14% decrease in pain in the study (“PA positioning induces current predominantly along the anterior-posterior axis of the brain). In fact, Andre-Obadia and others asserted that not only did LM rTMS not outperform PA rTMS, it did not outperform placebo stimulation either. The Andre-Obadia article teaches against the application of TMS with magnet configuration oriented perpendicular to the posterior-anterior axis of the head. On the basis of the work of Andre-Obadia (such as that published in the article mentioned above) and others, those of skill in the art have been attempting TMS using standard PA positioning of the coils, and avoiding transferse positioning. Although posterior-anterior (PA) positioning of the coil is typically used, a few references have described the use of an approximately 45% to the long axis of the head. (Brasil-Neto 1992, Mills et al 1992). In addition, a few articles in the literature do suggest a role in activation of nerve fibers by electrical currents that pass those fibers in a transverse manner. A review and theoretical consideration for the merits of the case for transverse activation are described in Ruohonen et al 1996. Ruohonen however, is a theoretical paper that does not teach specific methods for achieving improved analgesia or antidepressant effects using transverse positioning, nor the targeted modulation of deep-brain white matter tracts in this manner.

Thus, it would be desirable to achieve improved levels of analgesia with a minimum of side-effects, using transcranial magnetic stimulation. It would also be desirable to achieve improved antidepressant and other clinical effects using transcranial magnetic stimulation.

### SUMMARY

Described herein are methods for modulating brain activity of one or more target brain regions, the methods using low-frequency Transcranial Magnetic Stimulation (TMS) to provide significant analgesia. In particular, described herein are systems for arranging one or more (e.g. a plurality) of TMS electromagnet(s) is/are oriented in a transverse direction to the anterior-posterior (A-P) axis to the subject's head to provide significant analgesia. Lower powered coils within the array may be also placed in a direction transverse to the right/left midline axis of the head. As a result, the principal direction of the induced current from the TMS electromagnet(s) is/are oriented transverse to the A-P axis of the subject's head. This net effect is in contrast with standard TMS methods for treatment of pain and for depression that teach that electrical current should be induced in line with the A-P axis of the head. Surprisingly, when applying electromagnetic energy so that the induced current is primarily oriented transverse to the A-P axis of the subject's head, particularly in deep-brain stimulation, the net effect is a statistically significant effect (e.g., analgesia) compared to other orientations, which may not have a statistically significant effect. The methods and systems described herein are primarily multi-coil TMS systems. The orientation of the coils may be used to determine the aggregate effect and orientation on the deep brain target.

Applying either low-frequency (e.g., less than about 5 Hz, less than about 2 Hz, etc.) or high frequency stimulation (5 Hz or greater, for example 10 Hz) produces significant analgesia acutely. High frequency stimulation produces significant pain reduction in patients with a chronic pain condition.

In general, the methods of treatment described herein including methods of treating a patient by applying Transcranial Magnetic Stimulation (TMS). The method may include the steps of: positioning a plurality of TMS electromagnets outside of a subject's head towards a target brain region so that the principal direction of electrical current induced by the electromagnet is transverse to a long axis of a target nerve tract within the target brain region; and treating the patient by applying stimulation from the TMS electromagnet to the target brain region. The treatment may be directed to a therapeutic treatment such as the treatment of depression, the relief of pain, etc. The target may be a deep brain target, and may be a target associated with a desired therapeutic effect.

For example, described herein are methods of modulating brain targets such as the Dorsal Anterior Cingulate Gyrus (DACG) so as to produce robust analgesia by the application of low-frequency Transcranial Magnetic Stimulation (TMS) that include the steps of positioning a TMS electromagnet outside of a subject's head towards a target brain region so that the principal net direction of current flowing in the TMS electromagnet is transverse to the A-P axis of the subject's head; and evoking significant analgesia by impacting the target brain region(s) by applying a low-frequency stimulation from the TMS electromagnet.

The step of modulating brain activity may include applying a frequency below 5 Hz, or below 2 Hz (e.g., between about 0.5 and 2 Hz). The step of modulating brain activity may include applying a frequency above 5 Hz. Such high frequency TMS (for example trains of 10 Hz pulses) are of particular utility in treating chronic pain conditions, as well as depression.

In some variations, the step of positioning comprises positioning a plurality of TMS electromagnets outside of the subject's head towards the target brain region so that the principal direction of current in at least one of the TMS electromagnets is transverse to the A-P axis of the subject's head.

Also described herein are methods of producing analgesia by the application of low-frequency Transcranial Magnetic Stimulation (TMS), the method comprising: positioning a TMS electromagnet outside of a subject's head towards a target brain region so that the principal direction of current evoked by the TMS electromagnet is transverse to the A-P axis of the subject's head; and producing robust anesthesia by neuromodulating the target brain region by applying a stimulation from the TMS electromagnet. In some variations, the step of stimulating comprises applying a frequency of stimulation from the TMS electromagnet that is less than about 5 H, less than 2 Hz, etc.

For example, described herein are methods of reducing pain by the application of Transcranial Magnetic Stimulation (TMS). Such a method may include: positioning a TMS electromagnet outside of a subject's head towards a target brain region so that the principal direction of electrical current induced by the electromagnet is transverse to the anterior-posterior axis of the subject's head; and reducing pain levels by applying stimulation from the TMS electromagnet to the target brain region.

Reducing pain levels may comprise applying a frequency above about 5 Hz (e.g. 10 Hz, 10-20 Hz, etc.). The step of reducing pain levels may comprise applying a frequency below 2 Hz.

The step of positioning the TMS electromagnet may comprise positioning a plurality of electromagnets outside of the subject's head towards the target brain region so that the principal direction of current in at least one of the electromagnets is transverse to the anterior-posterior axis of the subject's head. A frame (e.g., gantry, clamp, arm, helmet, or other “holder”) may be used to hold the plurality of TMS electromagnets in position relative to the patient's head. The frame may be configured to allow adjustment (to each patient or to different target deep brain regions) of one or more of the TMS electromagnets, and may be further configured to lock or hold them in place for or during the application of energy.

Any appropriate target brain region may be chosen, particularly deep brain regions. For example, the target brain region may be the Dorsal Anterior Cingulate Gyrus. For example, the step of positioning the TMS electromagnet may comprise positioning the TMS electromagnet so that the principal direction of current in the electromagnets is transverse to the cingulate gyrus.

Another deep brain target region includes the medial forebrain bundle. The method may include positioning the TMS electromagnet by positioning the TMS electromagnet so that the principal direction of current in the electromagnets is transverse to the medial forebrain bundle.

Also described herein are methods of reducing pain by the application of Transcranial Magnetic Stimulation (TMS) using an array of TMS electromagnets arranged so that the induced current is transverse to the AP axis. For example, the method may include: positioning a plurality of TMS electromagnets outside of a subject's head towards a target deep brain region so that the principal direction of electrical current evoked by the electromagnets is transverse to the anterior-posterior axis of the subject's head; and reducing pain levels by applying energy from the electromagnet to the target deep brain region.

As mentioned, the method may include reducing pain levels by applying stimulation comprising applying a frequency of stimulation from the electromagnets that is above about 5 Hz (e.g., 10 Hz, between 10-20 Hz, etc.) and/or less than about 2 Hz.

Also described herein are Transcranial Magnetic Stimulation systems for deep brain stimulation comprising a 4-coil transverse array, the system comprising: a top TMS coil coupled to a frame configured to hold the top TMS coil anterior to Cz, wherein the top TMS coil is oriented so that primary current within the top TMS coil at the patient-contacting region of the top TMS coil is directed to the patient's right; a front TMS coil coupled to the frame, wherein the frame is configured to hold the front TMS coil near Fz, and oriented so that primary current within the front TMS coil is directed to the patient's left; and a left side TMS coil coupled to the frame, wherein the frame is configured to hold the left side TMS coil near F3 and oriented so that the primary current within the left side TMS coil is directed upwards; and a right side TMS coil coupled to the frame, wherein the frame is configured to hold the right side TMS coil near F4 and oriented so that the primary current within the right side TMS coil is directed upwards.

In some variations the system includes a three-coil array. For example, a system may include a Transcranial Magnetic Stimulation system for transverse stimulation of a patient's deep brain, the system comprising: a top TMS coil coupled to a frame configured to hold the top TMS coil anterior to Cz, wherein the top TMS coil is oriented so that primary current within the top TMS coil at the patient-contacting region of the top TMS coil is directed to the patient's right; a front TMS coil coupled to the frame, wherein the frame is configured to hold the front TMS coil near Fz, and oriented so that primary current within the front TMS coil is directed to the patient's left; and a side TMS coil coupled to the frame, wherein the frame is configured to hold the side TMS coil near F3 and oriented so that the primary current within the side TMS coil is directed upwards.

In general, the frame may be configured to hold the top, front and side TMS coils in a fixed orientation so that the principal direction of current in at least one of the top, front, and side TMS coils is transverse to the patient's medial forebrain bundle, whereby said medial forebrain bundle is stimulated.

As mentioned, the frame may be adjustable or fixed, and it may include articulating regions for moving the TMS coils out of the way or closer to the patient. The frame may be configured to hold the top, front and side TMS coils in a fixed orientation so that the principal direction of current in at least one of the top, front, and side TMS coils is transverse to the patient's cingulate bundle, whereby said cingulate bundle is stimulated.

Also described herein are methods of treating depression by the application of Transcranial Magnetic Stimulation (TMS), the method comprising: positioning a TMS electromagnet outside of a subject's head towards a target brain region so that the principal direction of electrical current induced by the electromagnet is transverse to the anterior-posterior axis of the subject's head; and reducing pain levels by applying stimulation from the electromagnet to the target brain region.

The step of positioning the TMS electromagnet may comprise positioning a plurality of TMS electromagnets outside of the subject's head towards the target brain region so that the principal direction of current in at least one of the electromagnets is transverse to the anterior-posterior axis of the subject's head. In some variations, positioning the TMS electromagnet comprises positioning a plurality of TMS electromagnets outside of the subject's head towards a target deep brain region. The step of positioning may include positioning a plurality of TMS electromagnets outside of the subject's head towards the target brain region so that the principal direction of current in at least one of the electromagnets is transverse to the cingulate bundle.

### BRIEF DESCRIPTION OF THE DRAWINGS

FIG. 1 illustrates conventional TMS coil positioned relative to a brain using a single figure-8 TMS coil oriented so it is parallel to the anterior-posterior axis of the head.

FIG. 2 illustrates TMS using an array of TMS coils arranged with the main axis of current along an anterior/posterior line.

FIG. 3 is a bar graph showing the analgesia effect resulting from employing the TMS configuration shown in FIG. 2.

FIG. 4 illustrates a traditional figure-8 TMS coil oriented so that it is transverse to the anterior-posterior axis of the head.

FIG. 5 illustrates transverse activation using an array of four double-coil TMS electromagnets.

FIG. 6 demonstrates robust analgesia resulting from employing the TMS configuration shown in FIG. 5.

FIG. 7 illustrates the location of the patient-contacting surfaces of the coils in one variation of a 4-coil transverse array, as related to a figure of a human head.

FIG. 8 shows the positions for each of the coils in the 4-coil transverse array positions on the head with respect to standard EEG-10-20 scalp locations.

FIG. 9 illustrates the location of the patient-contacting surfaces of the coils within a 3-coil transverse array, as related to a figure of a human head.

FIG. 10 shows the positions for each of the coils in the 3-coil transverse array positions on the head with respect to standard EEG-10-20 scalp locations.

FIG. 11 documents actual pain reduction results in patients with fibromyalgia comparing different array orientations.

FIG. 12 shows the acute pain reduction effect of a transverse 4-coil array when operated at 10 Hz, 3000 pulses in the orientation indicated herein.

FIG. 13 documents the antidepressant effect of the 4-coil transverse array operated at 10 Hz as compared with 45-degree multi-coil array.

FIG. 14 shows a table of magnetic field vectors associated with transverse coil arrays as compared with a standard single coil in standard (A/P-oriented) positioning. Values represent B-field power produced by 1 amp DC simulation input to each coil, and are directly proportional to the values that are produced when powering each coil with an actual TMS pulse

FIG. 15 identifies the axes referred to in FIG. 14 with respect to a human head.

### DETAILED DESCRIPTION

In general, described herein are TMS treatment systems, devices and methods for neuromodulation. In particular, the systems and methods described herein may be used to help treat pain (e.g., for analgesia) or depression. These systems and methods may be particularly configured for the neuromodulation of both superficial and/or deep-brain targets, including the dorsolateral prefrontal cortex, Dorsal Anterior Cingulate Gyms (cingulate gyrus), medial forebrain bundle, etc.

Although the inventors do not wish to be bound by any particular theory of operation, one mechanism by which the transverse arrays described herein might exert their effect is direct action of magnetic fields upon deep white matter tracts (such as the cingulate bundles or medial forebrain bundle) in response to transverse induced electrical currents. Such types of neuromodulation are described in greater detail for example, in U.S. patent application Ser. No. 12/324,227, titled “TRANSCRANIAL MAGNETIC STIMULATION OF DEEP BRAIN TARGETS”, incorporated by reference in its entirety.

As used herein, the term “deep brain” refers to the region of the patient's brain that deeper within the brain than the outer cortical regions of the brain. Although the outer cortical regions maybe stimulated using the methods, devices and systems described herein, the deep brain regions are of particular interest. Examples of deep brain regions may include (but are not limited to): subthalamic nucleus, globus pallidus extema, anterior cingulate gyrus, posterior cingulate gyms, subgenual cingulate gyms, anterior cingulate, dorsal cingulate gyrus, ventromedial nucleus of thalamus, ventrolateral nucleus of thalamus, anterior limb of the internal capsule, nucleus accumbens, septal nucleus, hippocampus, medial forebrain bundle, etc.

Although the examples and illustrations described herein typically include a plurality of TMS electromagnets (TMS coils), one, two, three, four or more TMS electromagnet coils may be used. The system may generally include a frame (e.g., a scaffold, holder, arm, gantry, or the like) to hold the one or more TMS electromagnets in position outside of a patient's head for TMS application so that the majority of the current evoked by the TMS coils is transverse to the anterior-posterior (A-P) axis of the patient's head. The frame may be adjustable, but may include preset locking positions for holding the TMS electromagnets in position to provide transverse current at or near the deep brain target.

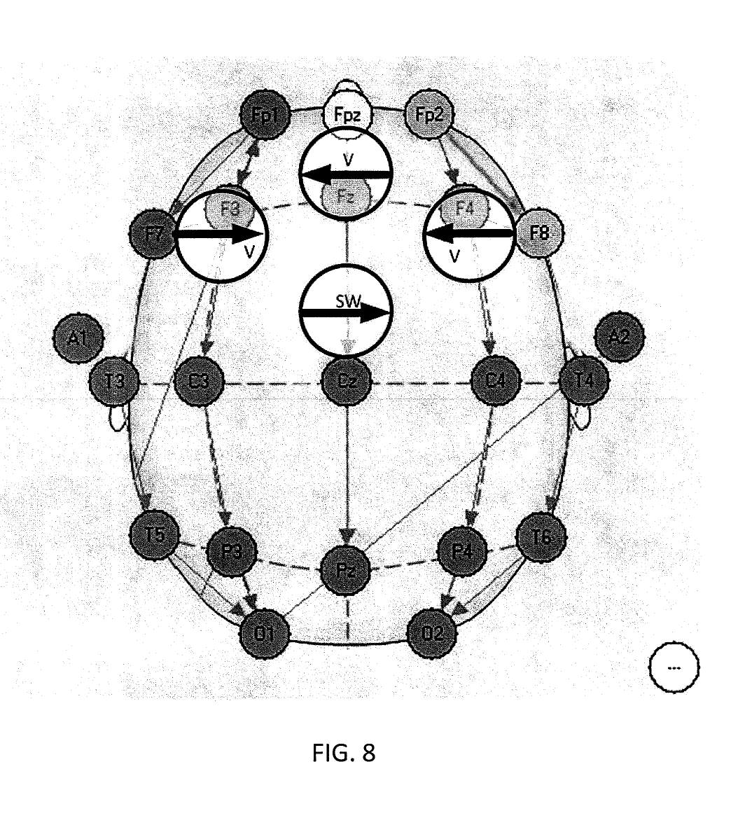

Any appropriate TMS electromagnet(s) may be used, including traditional “FIG. 8” TMS coils, as well as bent TMS coils, swept-wing TMS coils, V-shaped TMS coils, and the like. A bent and/or swept-wing TMS coil may include a plurality of coil windings that meet at a central region and extend outward from the central region (which may be “flat” in swept-wing embodiments) out of the plane of the central region of the magnet. Examples of such TMS coils are illustrated in PCT Application No. PCT/US2010/020324, titled “SHAPED COILS FOR TRANSCRANIAL MAGNETIC STIMULATION”, filed on Jan. 7, 2010, previously incorporated by reference in its entirety.

For example, described herein are systems and methods for modulating brain regions, including deep brain target regions, using one or more TMS electromagnets configured for low-frequency stimulation to achieve robust anesthesia by neuromodulating target brain regions. Some of the targets for treatment of pain are the Dorsal Anterior Cingulate Gyrus (DACG), the prefrontal cortex, and the motor cortex. Neuromodulation producing analgesia of targets such at the DACG may involve down-regulation at that target. In particular, as described herein, robust anesthesia caused by neuromodulation of target brain regions may be achieved using low or high-frequency TMS in which the primary direction of current in the TMS coil is perpendicular to the anterior-posterior axis of the head (e.g., the AP axis of the head, skull, brain, etc.). This configuration is called the transverse configuration.

In some variations, the methods of performing TMS to treat a patient include treatment at low frequency to neuromodulate the target brain regions by orienting the TMS coil so that the direction of the primary current in the TMS coil is transverse to the Anterior-Posterior axis of the head. (The direction of the electrical current induced by the TMS is typically opposite to the direction of the primary applied current within the coil windings, thus along the same axis). Thus, in the invention herein described, the direction of the induced electrical current within the brain is perpendicular to the AP axis of the head.

Low frequency stimulation may include stimulation at or below 5 Hz (e.g., 5 Hz, 1 Hz, etc.). In some variations the frequency may be greater than 10 Hz (e.g., between 10 Hz and 20 Hz, between 10 Hz and 50 Hz, etc.), which is termed herein “high-frequency” stimulation.

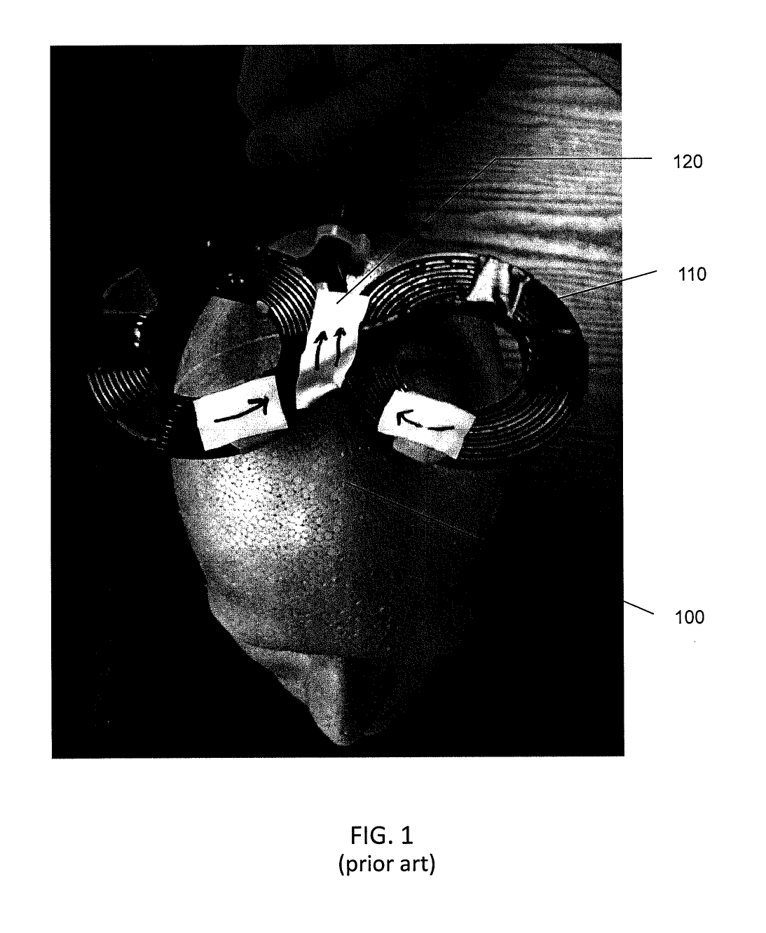

FIG. 1 illustrates a conventional TMS coil positioned relative to a brain using a single figure-8 TMS coil oriented so it is parallel to the anterior-posterior axis of the head. The main direction of the induced electrical currents (e.g., near the center region of the TMS coil) are in the direction of the A-P axis of the head.

In FIG. 1, patient-head representation 100 is shown with figure-8 double-coil 110 (without its shield so that actual coils can be viewed) with arrows 120 illustrating the current flow where both coils have, in the double-coil center, the current flowing in the same direction.

In FIG. 1, patient-head representation 100 is shown with figure-8 double-coil 110 (without its shield so that actual coils can be viewed) with arrows 120 illustrating the current flow where both coils have, in the double-coil center, the current flowing in the same direction. This example illustrates the standard and accepted method of positioning a TMS electromagnet. The electrical current induced in the target will be in opposite direction to arrows 120, along the A/P axis of the head. This is the traditional orientation in which TMS coils are used over patients' heads, and is used as a figure in order to illustrate the prior art.

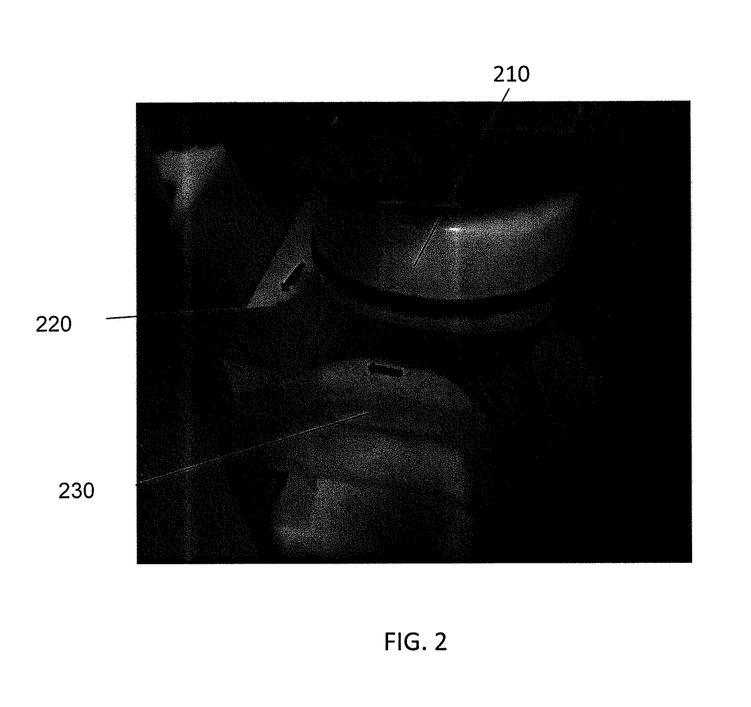

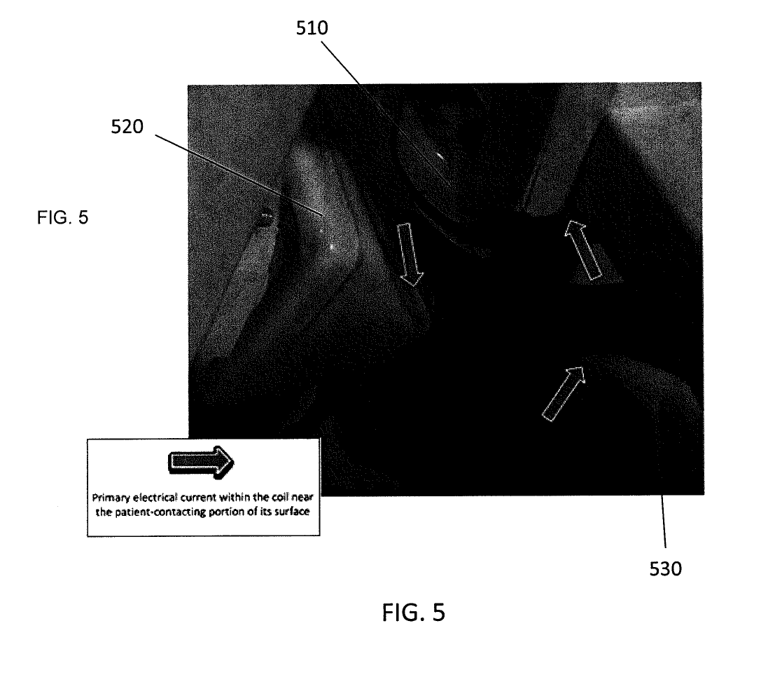

FIG. 2 shows an array of four TMS electromagnets oriented approximately along the A-P axis of the head. In FIG. 2, the primary directions of current (e.g., near the center regions of the TMS coils) are in the direction of the A-P axis of the head. The array of TMS electromagnets in FIG. 2 shows four double-coil TMS electromagnets of different configurations, including V-shaped or swept-wing coils. The four electromagnet coils are the swept-wing top double-coil 210, V-double-coil front coil 220, and V-double-coil side coil 230. Opposite V-double-coil side coil 230 is a companion V-double-coil side coil. In FIG. 2, the arrows shown on the TMS electromagnets indicate the primary direction of electrical current in each double coil structure at their geometric centers. In some examples, when the TMS magnets are oriented with the main axis of current (shown by arrows on each TMS coil in FIG. 2) oriented along an anterior/posterior line, stimulation (e.g., 1 Hz) results in down-regulation of affected brain tissue. As described in greater detail below, this A/P-oriented configuration was found to be surprisingly less effective for achieving analgesia when used to stimulate particular deep-brain target regions. Instead, configurations in which one or more of the TMS electromagnets (or the net effect of the TMS electromagnets) results in transverse current at the deep brain target region, e.g., transverse to the A-P axis showed superior performance in inducing analgesia, and better ability to reach the deep dorsal anterior cingulate region in brain imaging studies. For example, the orientation described below for FIG. 5 was surprisingly superior in performance compared to the arrangement illustrated in FIG. 2.

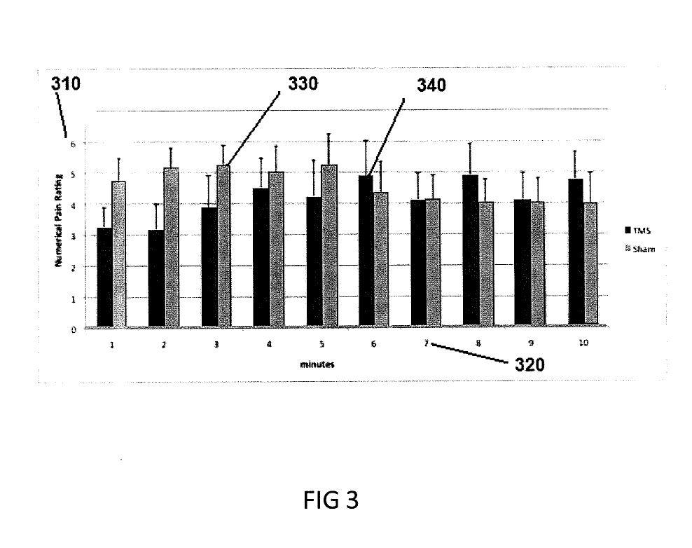

FIG. 3 illustrates the clinical analgesic effect of slow-rate stimulation in the configuration shown in FIG. 2 on various target structures affected by the TMS after a pain stimulus was applied. To do the pain study, the skin of the subject is first sensitized with capsaicin. This is followed by the heat threshold and tolerance being determined with Peltier thermode over the sensitized area. Then pain a stimulus is administered with a Peltier thermode at constant temperature over sensitized area where the stimulus corresponds with 60% tolerance level. Verbal reports on pain level were obtained every one minute for ten minutes. In this example, a subject was either stimulated using low-frequency stimulation or was sham stimulated. For an average of seven subjects, the Numerical Pain Rating on a scale of 2 to 10 as overall average was an approximately 30% reduction in reported pain. Overall, the activity in the target brain regions (e.g., DACG, which usually becomes more active in the presence of pain) was lowered following real versus sham magnetic stimulation, indicating a down-regulation in activity, and this down-regulation was accompanied by significant analgesia.

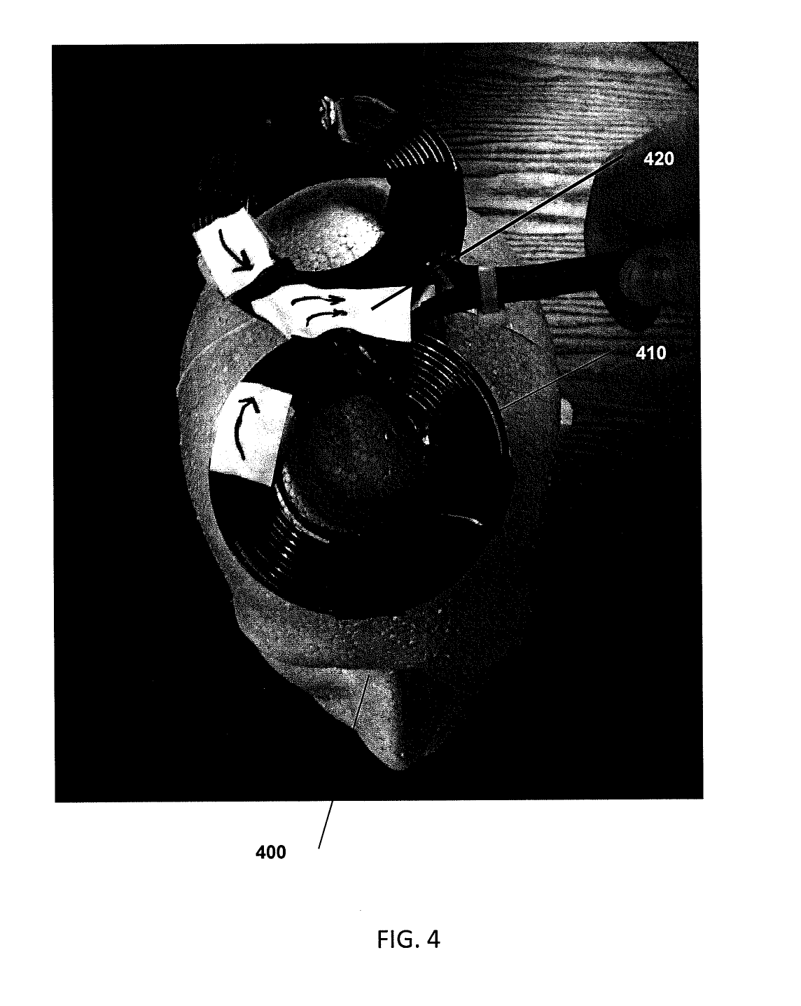

In contrast, the present invention achieved substantially more clinical analgesia when the orientation of the TMS electromagnet(s) is/are rotated by approximately 90 degrees, so that the principal direction of the current in the TMS electromagnet(s) is oriented transverse to the A-P axis of the head. FIG. 4 illustrates a traditional figure-8 TMS coil oriented so that it is transverse to the anterior-posterior axis of the head. The primary direction of the currents (e.g., near the center region of the TMS coil, is transverse to the direction of the A-P axis of the head. In FIG. 4, patient-head representation 400 is shown with figure-8 double-coil 410 (without its shield so that actual coils can be viewed) with arrows 420 illustrating the current flow where both coils have, in the double-coil center, the current flowing in the same direction. The electrical current induced in the target will be in opposite direction to arrows 420.

In FIG. 5 transverse activation is illustrated using a four double-coil array. The four electromagnet coils are the swept-wing top double-coil 510, V-double-coil front coil 520, and V-double-coil side coil 530. Opposite V-double-coil side coil 530 is a companion V-double-coil side coil. Arrows indicate the main direction of primary electrical current in each double coil structure at their geometric centers. Slow-rate stimulation while in this novel orientation (e.g., at 1 Hz) results in robust analgesia due to neuromodulation of affected brain tissue, when compared to the analgesia obtained with the multiple-coil array in the AP configuration.

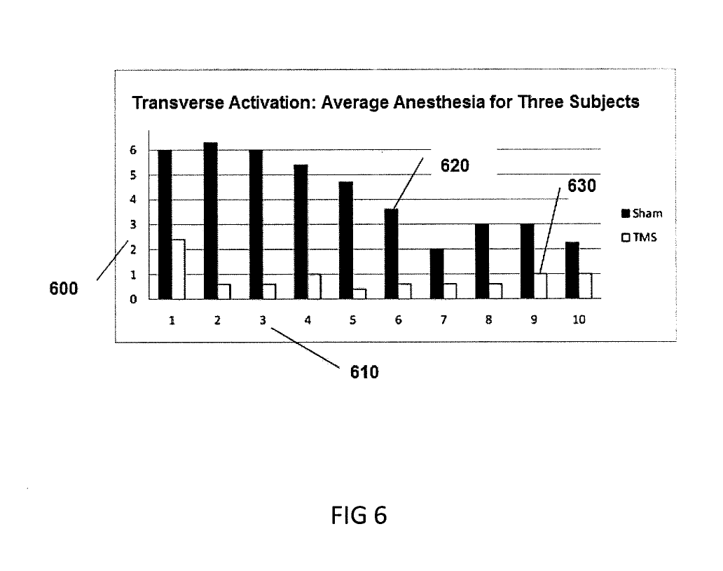

FIG. 6 illustrates the 60 to 94% reduction in pain obtained in studies of three subjects using the transverse orientation. In FIG. 6, Numerical Pain Score Rating scale 600 is used to evaluate pain levels 620 for Sham stimulation and 630 for TMS stimulation where the pain measurements were taken minute-by-minute over a ten-min period with the minutes marked in 610. The pain reduction in this transverse case is significantly greater than the 30% reduction shown in FIG. 3 where the coil array was oriented parallel to the anterior-posterior axis of the head.

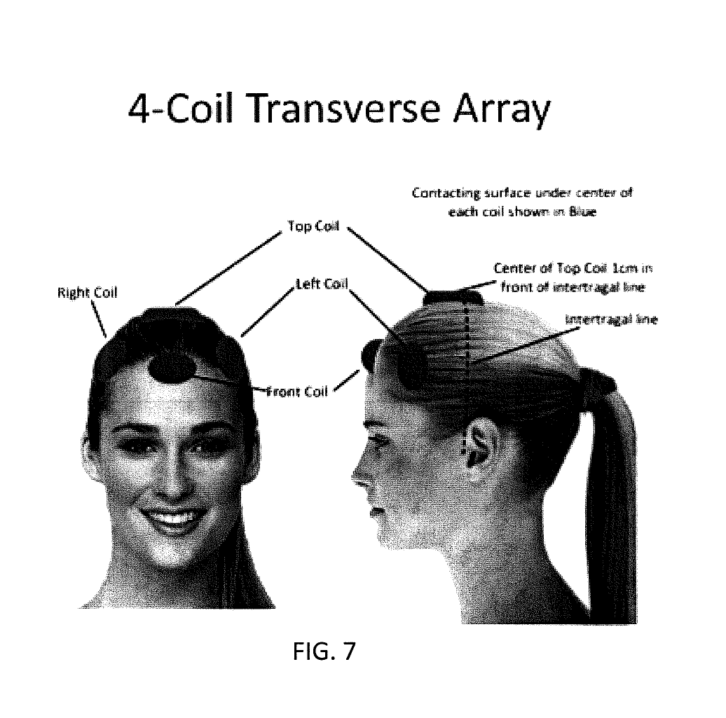

The arrangements of TMS coils used to achieve the results illustrated in FIG. 6 are shown below. In this example, an array of TMS coils of various types (swept-arm and V-shaped TMS coils) are arranged around the patient's head and held in place using a frame that holds the coils so that the primary induced current from one or all of the TMS coils will be oriented (as confirmed by simulation of the applied fields) transverse to the A-P axis of the patient's head. For example, FIG. 7 illustrates the locations of the patient-contacting surfaces of four coils within a 4-coil transverse array, as related to a figure of a human head. The shortest line between the tragic of the ears on each side of the head may be used as a guide to center the top coil forward of that line so as to avoid inadvertent motor cortex stimulation. Placement of the TMS coils using this arrangement generally may be modified depending on the particular deep-brain target region. For example, the deep-brain target region may be oriented for transverse deep-brain stimulation of the deep-brain target.

FIG. 8 shows the positions for each of the coils in the 4-coil transverse array positions on the head (as shown in FIG. 7) with respect to standard EEG-10-20 scalp locations. The large circles represent each of the four coils in the array. For example, the top coil may be of a swept-wing (“SW”) design, and be located anterior to Cz, anterior and clear of motor cortex. Left and right side coils may be of V-coil design (“V”) and be placed approximately at F3 and F4, respectively. A front coil may also be of the V design and be placed approximately over Fz. The arrow within each coil representation circle may indicate the direction of the primary electrical current within the coil near the patient-contacting portion of its surface. The effect of such a coil is generally to drive induced electrical current within the brain in a direction opposite of primary electrical current. In this transverse 4-coil array, the primary current within the coil at the patient-contacting (center) of the top coil is directed to the patient's right; the front coil to the patient's left, and the left and right side coils, upward. In FIG. 8, the large circles represent scalp-contacting surface of coil centers, as mentioned, arrows in the circles represent the direction of primary electrical current within the coil near the patient contacting portion of its surface

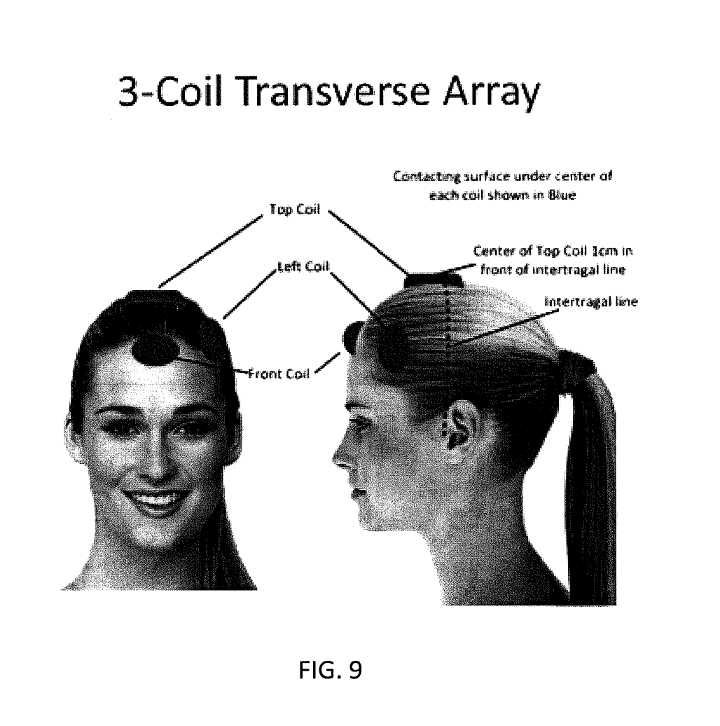

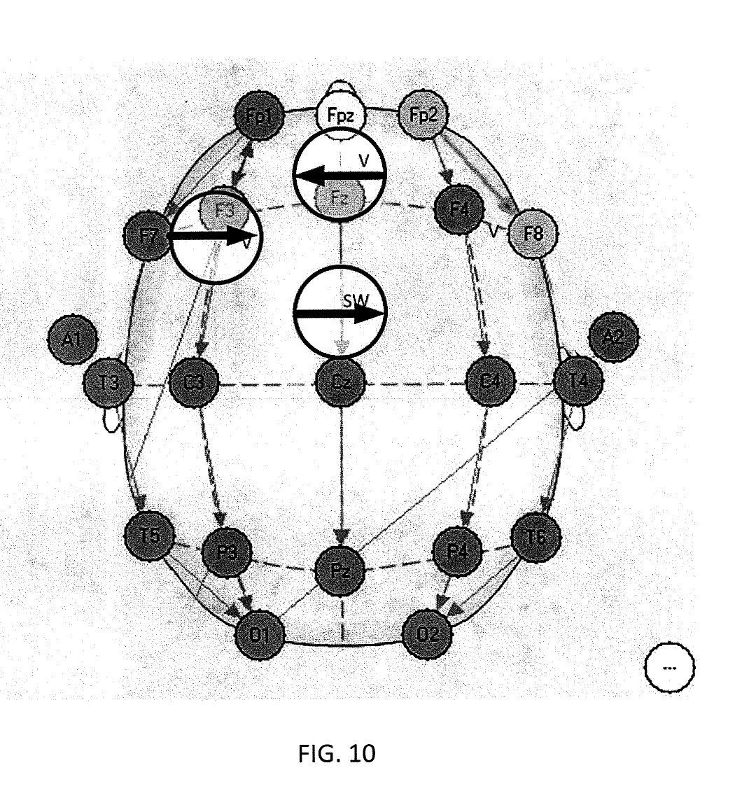

Another alternative is shown in FIG. 9. FIG. 9 illustrates the location of the patient-contacting surfaces of the coils within a 3-coil transverse array, as related to a figure of a human head. The shortest line between the tragic of the ears on each side of the head may be used as a guide to center the top coil forward of that line so as to avoid inadvertent motor cortex stimulation. FIG. 10 shows the positions for each of the coils in the 3-coil transverse array positions of FIG. 9 on a head with respect to standard EEG-10-20 scalp locations. The large circles represent each of the four coils in the array. In this example, the top coil may be of a swept-wing (“SW”) design, and be located anterior to Cz, anterior and clear of motor cortex. Left side coil (or alternatively a right side coil) may be of V-coil design (“V”) and be placed approximately at F3. The front coil may also be of the V design and be placed approximately over Fz. The arrow within each coil representation circle indicates the direction of the primary electrical current within the coil near the patient-contacting portion of its surface. The effect of such a coil is generally to drive induced electrical current within the brain in a direction opposite of primary electrical current. In this transverse 3-coil array, the primary current within the coil at the patient contacting (center) of the top coil is directed to the patient's right; the front coil to the patient's left, and the left side coil is upward.

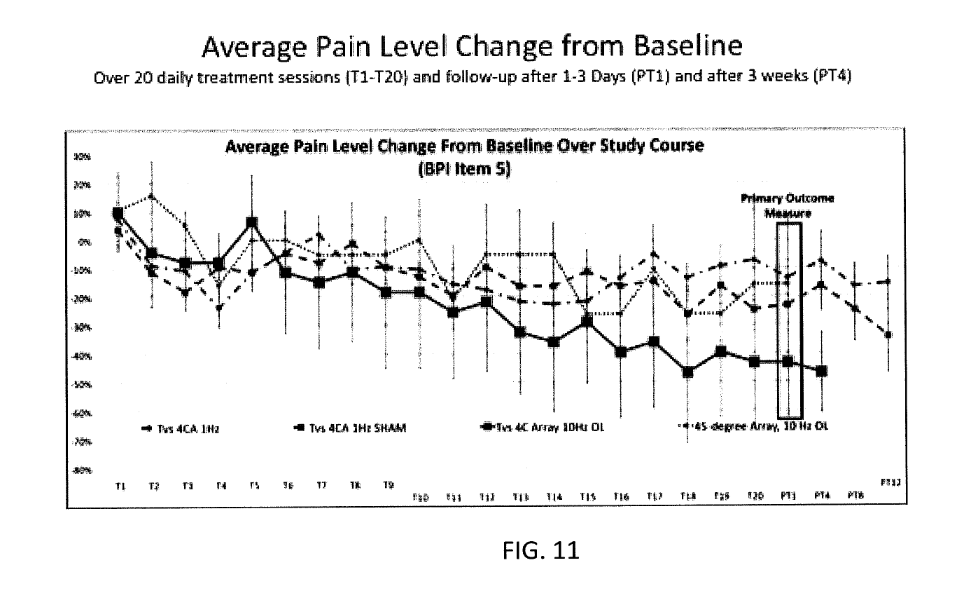

FIG. 11 documents actual pain reduction results in patients with fibromyalgia, a chronic disease condition in which pain is a significant feature. “Average pain level over the last 24 hours” is reflected in the score of Item 5 in the standard Brief Pain Inventory. Over a series of 20 TMS treatment sessions and follow-up period, the scores from each of four different treatment groups are shown as averages for that group. The first group is shown with small dashed lines, and refers to patients for whom receiving traditional stimulation (not transverse to the AP axis) at 1 Hz. The second group, with alternating short dashed lines and dots is from patients receiving SHAM stimulation (at 1 Hz). The third group, represented by a thick black line and heavy square boxes, represents treatment with a transverse 4-coil array energized with 10 Hz pulse trains using the same traditional orientation of the array of TMS electromagnets. Finally, the fourth group, represent by a line consisting of dots only is an open-label test of a multi-coil array in which coils are turned to a 45-degree angle with respect to the anterior-posterior axis of the head, and energized with 10 Hz pulse trains. While all groups show reduction in pain levels relative to baseline (including the sham), only the line for the transverse 4-coil array operated at 10 Hz surpasses 30% pain reduction line (an industry standard for clinical utility), and reaches an average 42% reduction at the time of the pre-designated primary outcome measure. As shown in FIG. 11, this improvement is maintained and possibly improved 3 weeks later at post-treatment visit (PT) 2.

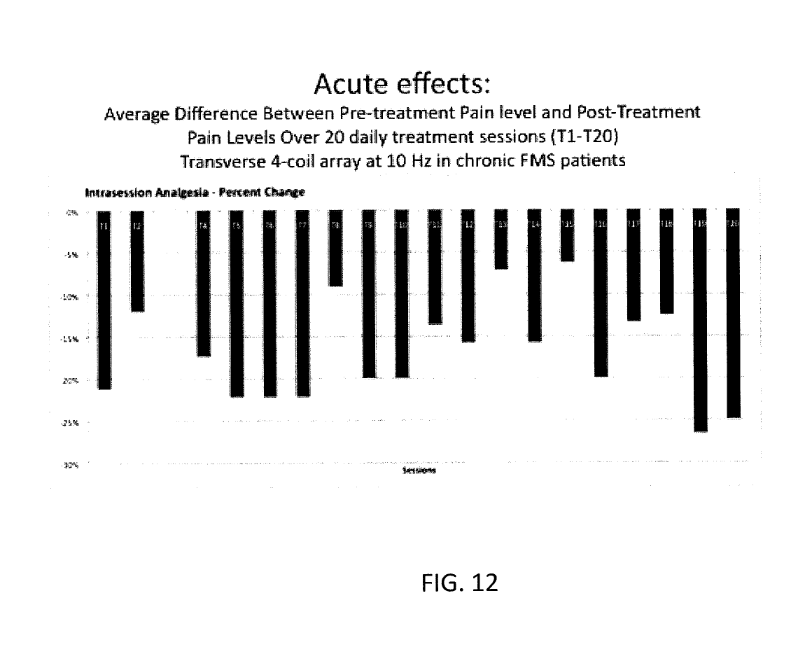

The acute effect of stimulation using this transverse orientation (at 10 Hz) is illustrated in FIG. 12. FIG. 12 is a corollary to the graph shown in FIG. 3, above. While FIG. 3 looked at acute pain reduction when the transverse array was operated at 1 Hz, FIG. 12 shows the acute pain reduction effect of a transverse 4-coil array when operated at 10 Hz, 3000 pulses.

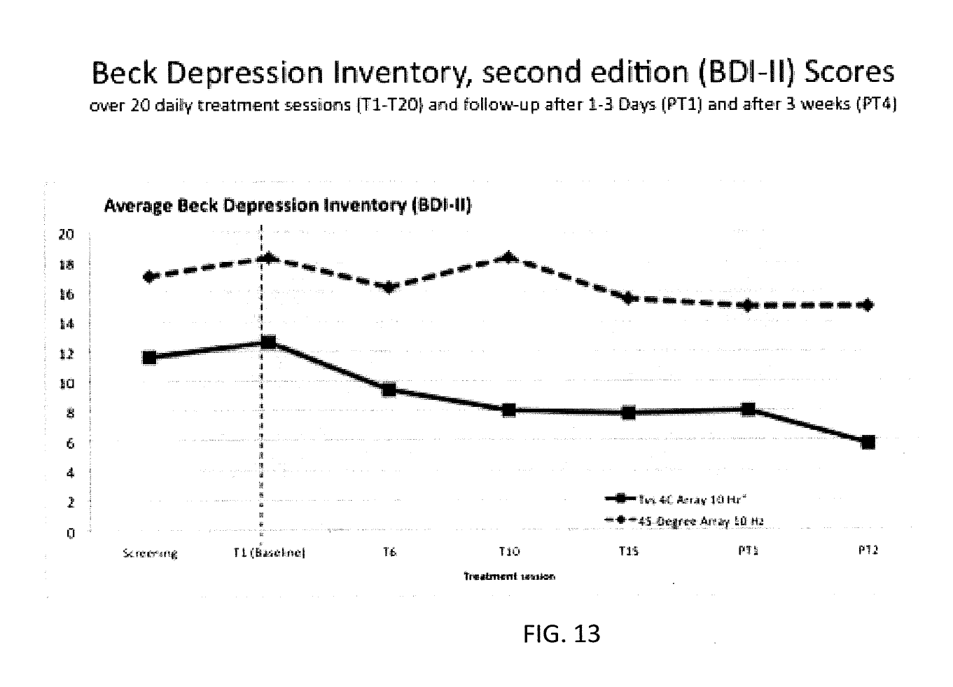

Although the data above examined the effect of transverse deep brain stimulation on pain (e.g., acute and chronic pain), other deep brain targets and effects may similarly be achieved using this configuration. Another indication that may be treated includes depression. For example, FIG. 13 documents the antidepressant effect of a 4-coil transverse array operated at 10 Hz as compared with 45-degree multi-coil array as measured by the Beck Depression Inventory, second edition (BDI-II). The transverse array shows a significant reduction in BDI-II scores, but the 45-degree array shows a non-significant reduction in the depression score.

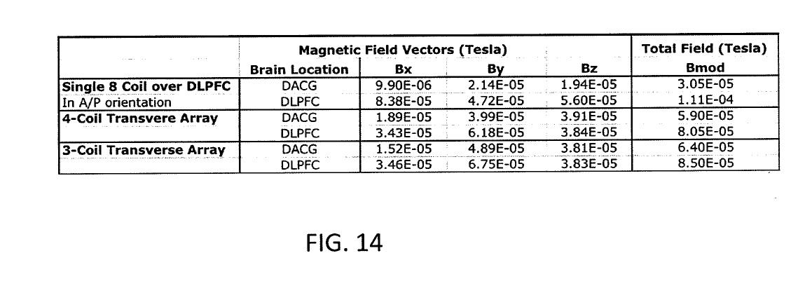

FIG. 14 shows a table of magnetic field vectors associated with transverse coil arrays as compared with a standard single coil in standard (A-P oriented) positioning. Values represent B-field power produced by 1 amp DC simulation input to each coil, and are directly proportional to the values that are produced when powering each coil with an actual TMS pulse generator, but of much smaller magnitude. However for any given power level or type delivered to these coils, the relationship between the magnitudes of the magnetic field at those positions is space will be the same. Specifically, note that the 4-coil transverse array and the 3-coil transverse array both deliver more power to the dorsal anterior cingulate (DACG) than does a standard figure-8 coil placed over the DLPFC. Also note that the three-coil array contains more B-field power in the Y vector, but that the 4-coil array produces almost as much in the Y, and more in Z axis than its 3-coil cousin. Note that the positive or negative values in the B-field vectors (X, Y, Z) are significant in that they add and subtract from one another. However, because the coils used with the present invention are frequently biphasic, these polarities will reverse one or more times during the firing of a pulse from each coil.

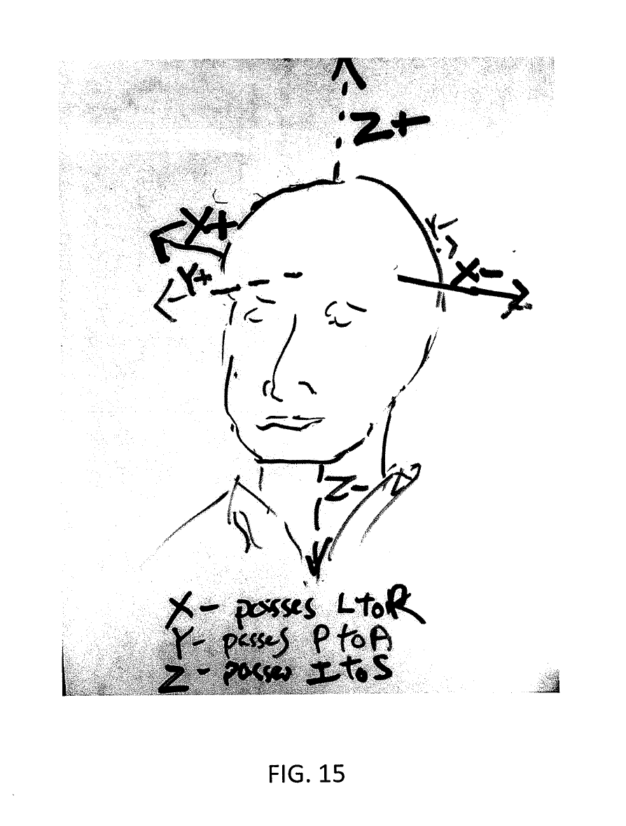

FIG. 15 Illustrates the directions of the X, Y and Z coordinates as used in the FIG. 14 table. In summary, X is in the coronal plane, passing from left (negative) to right (positive). Y is in the sagittal plane, passing from posterior (negative) to anterior (positive). Z is in the axial plane, passing from inferior (negative) to superior (positive).

Thus, in general, the methods described herein may be used to apply stimulation (including low-frequency stimulation) using a TMS system to reduce pain via positioning the TMS electromagnet(s) is/are oriented so that the principal direction of current in the TMS electromagnet is transverse to the AP axis of the subject's head (brain). The principal direction of current in the TMS electromagnet may be the net direction of current, or it may be the direction of current in the geometric center of the TMS electromagnet, particularly in TMS electromagnets having dual coils. Any appropriate TMS electromagnet configuration may be used, including, but not limited to, figure-8 coils that are flat, bent or curved, V-shaped TMS electromagnets, swept-wing or flat-bottomed TMS electromagnets, or the like. Examples of different TMS electromagnet configurations may be found in PCT application PCT/US2008/075706 (WO 2009/033192), titled “FOCUSED MAGNETIC FIELDS” (filed Sep. 9, 2008), and in PCT Application No. PCT/US2010/020324, titled “SHAPED COILS FOR TRANSCRANIAL MAGNETIC STIMULATION”, filed on Jan. 7, 2010.

In some variations, one, some, or all of the TMS electromagnets are oriented transverse to the AP axis of the subject's head when applying low-frequency TMS. In some variations the direction of the net induced current from the plurality of TMS electromagnets is transverse to the AP axis.

As used herein, low-frequency TMS may be used synonymously with slow rate or slow rTMS pulse rates, and may be between about 0.5 Hz to about 2 Hz. This low-frequency or slow rate rTMS pulse rates may be contrasted with “fast” rTMS pulse rates (e.g., between about 5-50 Hz). Thus, in some variations, low-frequency TMS may be less than about 5 Hz.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

What is claimed is:

1. A method of reducing pain by the application of Transcranial Magnetic Stimulation (TMS), the method comprising:
positioning a TMS electromagnet outside of a subject's head towards a target brain region so that the principal direction of electrical current induced by the electromagnet is transverse to the anterior-posterior axis of the subject's head; and
reducing pain levels by applying stimulation from the TMS electromagnet to the target brain region.

2. The method of claim 1, wherein reducing pain levels comprises applying stimulation at a frequency above about 5 Hz.

3. The method of claim 1, wherein reducing pain levels comprises applying stimulation at a frequency below 2 Hz.

4. The method of claim 1, wherein positioning the TMS electromagnet comprises positioning a plurality of electromagnets outside of the subject's head towards the target brain region so that the principal direction of current in at least one of the electromagnets is transverse to the anterior-posterior axis of the subject's head.

5. The method of claim 1, wherein the target brain region is a deep brain region.

6. The method of claim 1, wherein the target brain region is the Dorsal Anterior Cingulate Gyrus.

7. The method of claim 1, wherein positioning the TMS electromagnet comprises positioning the TMS electromagnet so that the principal direction of current in the electromagnets is transverse to the cingulate gyms.

8. The method of claim 1, wherein positioning the TMS electromagnet comprises positioning the TMS electromagnet so that the principal direction of current in the electromagnets is transverse to the medial forebrain bundle.

9. A method of reducing pain by the application of Transcranial Magnetic Stimulation (TMS), the method comprising:
positioning a plurality of TMS electromagnets outside of a subject's head towards a target deep brain region so that the principal direction of electrical current evoked by the electromagnets is transverse to the anterior-posterior axis of the subject's head; and
reducing pain levels by applying energy from the electromagnet to the target deep brain region.

10. The method of claim 9, wherein reducing pain levels by applying stimulation comprises applying stimulation at a frequency of stimulation from the electromagnets that is above about 5 Hz.

11. The method of claim 9, wherein reducing pain levels by applying stimulation comprises applying stimulation at a frequency of stimulation from the electromagnet that is less than about 2 Hz.

12. The method of claim 9, wherein the target deep brain region is the Dorsal Anterior Cingulate Gyrus.

13. The method of claim 9, wherein reducing pain levels by applying stimulation comprises transverse stimulation of a fiber bundle with a magnetic coil array.

14. The method of claim 9, wherein positioning the plurality of TMS electromagnets comprises positioning the TMS electromagnets so that the principal direction of current in the electromagnets is transverse to the cingulate gyms.

15. The method of claim 9, wherein positioning the plurality of TMS electromagnets comprises positioning the TMS electromagnets so that the principal direction of current in at least one of the electromagnets is transverse to the medial forebrain bundle.

16. A Transcranial Magnetic Stimulation system for deep brain stimulation comprising a 4-coil transverse array, the system comprising:
a top TMS coil coupled to a frame configured to hold the top TMS coil anterior to Cz, wherein the top TMS coil is oriented so that primary current within the top TMS coil at the patient-contacting region of the top TMS coil is directed to the patient's right;
a front TMS coil coupled to the frame, wherein the frame is configured to hold the front TMS coil near Fz, and oriented so that primary current within the front TMS coil is directed to the patient's left; and
a left side TMS coil coupled to the frame, wherein the frame is configured to hold the left side TMS coil near F3 and oriented so that the primary current within the left side TMS coil is directed upwards; and
a right side TMS coil coupled to the frame, wherein the frame is configured to hold the right side TMS coil near F4 and oriented so that the primary current within the right side TMS coil is directed upwards.

17. A Transcranial Magnetic Stimulation system for transverse stimulation of a patient's deep brain, the system comprising:
a top TMS coil coupled to a frame configured to hold the top TMS coil anterior to Cz, wherein the top TMS coil is oriented so that primary current within the top TMS coil at the patient-contacting region of the top TMS coil is directed to the patient's right;
a front TMS coil coupled to the frame, wherein the frame is configured to hold the front TMS coil near Fz, and oriented so that primary current within the front TMS coil is directed to the patient's left; and
a side TMS coil coupled to the frame, wherein the frame is configured to hold the side TMS coil near F3 and oriented so that the primary current within the side TMS coil is directed upwards.

18. The system of claim 17, wherein the frame is configured to hold the top, front and side TMS coils in a fixed orientation so that the principal direction of current in at least one of the top, front, and side TMS coils is transverse to the patient's medial forebrain bundle, whereby said medial forebrain bundle is stimulated.

19. The system of claim 17, wherein the frame is configured to hold the top, front and side TMS coils in a fixed orientation so that the principal direction of current in at least one of the top, front, and side TMS coils is transverse to the patient's cingulate bundle, whereby said cingulate bundle is stimulated.

20. A method of treating depression by the application of Transcranial Magnetic Stimulation (TMS), the method comprising:
positioning a TMS electromagnet outside of a subject's head towards a target brain region so that the principal direction of electrical current induced by the electromagnet is transverse to the anterior-posterior axis of the subject's head; and
reducing pain levels by applying stimulation from the electromagnet to the target brain region.

21. The method of claim 20, wherein positioning the TMS electromagnet comprises positioning a plurality of TMS electromagnets outside of the subject's head towards the target brain region so that the principal direction of current in at least one of the electromagnets is transverse to the anterior-posterior axis of the subject's head.

22. The method of claim 20, wherein positioning the TMS electromagnet comprises positioning a plurality of TMS electromagnets outside of the subject's head towards a target deep brain region.

23. The method of claim 20, wherein positioning comprises positioning a plurality of TMS electromagnets outside of the subject's head towards the target brain region so that the principal direction of current in at least one of the electromagnets is transverse to the cingulate bundle.

24. A method of treating a patient by applying Transcranial Magnetic Stimulation (TMS), the method comprising:
positioning a plurality of TMS electromagnets outside of a subject's head towards a target brain region so that the principal direction of electrical current induced by the electromagnet is transverse to a long axis of a target nerve tract within the target brain region; and
treating the patient by applying stimulation from the TMS electromagnet to the target brain region.

25. The method of claim 24, wherein treating comprises applying stimulation at a frequency above about 5 Hz.

26. The method of claim 24, wherein treating comprises applying stimulation at a frequency below 2 Hz.

27. The method of claim 24, wherein the target brain region is a deep brain region.

28. The method of claim 24, wherein the target brain region is the Dorsal Anterior Cingulate Gyrus.

29. The method of claim 24, wherein positioning the plurality of TMS electromagnets comprises positioning the TMS electromagnets so that the principal direction of current in the electromagnets is transverse to the cingulate gyms.
